(19) United States
(12) Patent Application Publication
(10) Pub. No.: US 2005/0240124 A1
(43) Pub. Date: Oct. 27, 2005

(54) Ultrasound medical treatment system and method

(76) Inventors: T. Douglas Mast, Cincinnati, OH (US); Waseem Faidi, Clifton Park, OH (US); Inder Raj S. Makin, Loveland, OH (US); Megan M. Runk, Cincinnati, OH (US); Michael H. Slayton, Tempe, AZ (US); Peter G. Barthe, Phoenix, AZ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/825,090

(22) Filed: Apr. 15, 2004

(51) Int. Cl. A61B 8/00; A61H 1/00

(52) U.S. Cl. 601/2; 600/439

(57) ABSTRACT

An ultrasound medical treatment system includes an ultrasound medical treatment transducer and a controller. In one arrangement, the controller movingly controls the medical treatment transducer to emit ultrasound to thermally ablate patient tissue: 1) for a plurality of predetermined time intervals each associated with the medical treatment transducer movingly disposed at a different one of an equal number of predetermined positions, wherein a next-in-time time interval is associated with a position which is spatially non-adjacent to a position associated with a present-in-time time interval; or 2) for a predetermined time interval during which the transducer is continuously moved. Methods of the invention so control the medical treatment transducer using or not using the controller. In another arrangement, the transducer has an array of transducer elements and the controller activates different non-overlapping groups or different overlapping groups of transducer elements at different times.

### FIELD OF THE INVENTION

The present invention relates generally to ultrasound, and more particularly to an ultrasound medical treatment system and method.

### BACKGROUND OF THE INVENTION

Known ultrasound medical-treatment systems and methods include using ultrasound imaging (at low power) of patients to identify patient tissue for medical treatment and include using ultrasound (at high power) to ablate identified patient tissue by heating the tissue. In one arrangement, an ultrasound medical-imaging-and-treatment transducer performs imaging and treatment at separate times. In another arrangement, an ultrasound medical-imaging transducer and a separate ultrasound medical treatment transducer are used. A transducer can have one transducer element or an array of transducer elements.

In one procedure for ablating large tissue volumes with ultrasound, the ultrasound medical treatment transducer is stepwise translated along the transducer's longitudinal axis to spatially-adjacent translational positions (such as 1 centimeter, 3 centimeters, 5 centimeters, 7 centimeters, 9 centimeters, etc.) with ultrasound emitted for a lengthy predetermined time interval at each translational position relative to a much shorter step time to move to a next translational position. In another procedure, the ultrasound medical treatment transducer is stepwise rotated about the transducer's longitudinal axis to spatially-adjacent angular positions (such as 0 degrees, 20 degrees, 40 degrees, 60 degrees, 80 degrees, etc.) with ultrasound emitted for a lengthy predetermined time interval at each rotational position relative to a much shorter step time to move to a next rotational position. In an additional procedure, the emitted ultrasound medical-treatment beam is electronically or mechanically focused at different distances from the transducer corresponding to different treatment depths within patient tissue and/or steered to different beam angles.

Known ultrasound medical systems and methods include deploying an end effector having an ultrasound transducer (powered by a controller) outside the body to break up kidney stones inside the body, endoscopically inserting an end effector having an ultrasound transducer in the rectum to medically destroy prostate cancer, laparoscopically inserting an end effector having an ultrasound transducer in the abdominal cavity to medically destroy a cancerous liver tumor, intravenously inserting a catheter end effector having an ultrasound transducer into a vein in the arm and moving the catheter to the heart to medically destroy diseased heart tissue, and interstitially inserting a needle end effector having an ultrasound transducer needle into the tongue to medically destroy tissue to reduce tongue volume to reduce snoring.

Still, scientists and engineers continue to seek improved ultrasound medical treatment systems and methods.

### SUMMARY OF THE INVENTION

One expression of an embodiment of an ultrasound medical treatment system includes an ultrasound medical treatment transducer and a controller. The controller positionally controls the medical treatment transducer to emit ultrasound to thermally ablate patient tissue for a plurality of predetermined time intervals each associated with the medical treatment transducer positionally disposed at a different one of an equal number of predetermined positions, wherein a next-in-time time interval is associated with a position which is spatially non-adjacent to a position associated with a present-in-time time interval. A method of the invention so controls the medical treatment transducer using or not using the controller.

Another expression of an embodiment of an ultrasound medical treatment system includes an ultrasound medical treatment transducer and a controller. The controller positionally controls the medical treatment transducer to emit ultrasound to thermally ablate patient tissue for a predetermined time interval during which the medical treatment transducer substantially-continuously changes position. A method of the invention so controls the medical treatment transducer using or not using the controller.

An additional expression of an embodiment of an ultrasound medical treatment system includes an ultrasound medical treatment transducer and a controller. The medical treatment transducer has an array of ultrasound transducer elements and has a multiplicity of element groups each including at least one ultrasound transducer element of the array. Each ultrasound transducer element of the array belongs to only one element group. The controller controls the medical treatment transducer to emit ultrasound to thermally ablate patient tissue for a plurality of predetermined time intervals each associated with emitting ultrasound from a different one of the element groups.

A further expression of an embodiment of an ultrasound medical treatment system includes an ultrasound medical treatment transducer and a controller. The medical treatment transducer has an array of ultrasound transducer elements, wherein the ultrasound transducer elements are positioned substantially along a straight or curved line. The controller controls the medical treatment transducer to emit ultrasound to thermally ablate patient tissue by sequentially-in-time activating positionally-overlapping groups of sequential-in-position ultrasound transducer elements.

Several benefits and advantages are obtained from one or more of the expressions of the embodiment and/or the methods of the invention. Applicants found having temporally-adjacent ablation time intervals be associated with spatially non-adjacent transducer positions substantially avoids or reduces transient, ultrasound-caused, ultrasound-attenuating effects (e.g., from tissue cavitation, tissue boiling, and/or temperature-related increases in tissue ultrasonic absorption) found near conventionally stepwise just-treated spatially adjacent tissue. This increased treatment depth and achieved a more uniform thermal lesion.

Applicants also found substantially-continuously moving the ultrasound medical treatment transducer substantially avoids or reduces transient, ultrasound-caused, ultrasound-attenuating effects (e.g., from tissue cavitation, tissue boiling, and/or tissue temperature-related increases in ultrasonic absorption) found near conventionally stepwise just-treated spatially adjacent tissue. This increased treatment depth and achieved a more uniform thermal lesion.

Applicants believe that using different transducer element groups (of a medical treatment transducer having an array of transducer elements) for predetermined time intervals, wherein each element belongs to only one element group, or sequentially-in-time activating positionally-overlapping groups of sequential-in-position ultrasound transducer elements, should also substantially avoid or reduce transient, ultrasound-caused, ultrasound-attenuating effects (e.g., from tissue cavitation, tissue boiling and/or temperature-related increases in tissue ultrasonic absorption) found near conventionally stepwise just-treated spatially adjacent tissue. This should increase treatment depth and achieve a more uniform thermal lesion.

Thus, one or more of the methods or expressions of the embodiment of the invention should result in more consistent lesion size and quality across different tissue properties, geometries, and ultrasonic source conditions, and the resulting reduction of ultrasound-attenuating effects (e.g., screening and shadowing ultrasound effects) should allow greater treatment depths, shorter treatment times, and/or the formation of more regular and controllable (and therefore more spatially selective) thermal lesions.

The present invention has, without limitation, application in conventional extracorporeal, endoscopic, laparoscopic, intra-cardiac, intravenous, interstitial and open surgical instrumentation as well as application in robotic-assisted surgery.

### BRIEF DESCRIPTION OF THE FIGURES

FIG. 1 is a schematic view of an embodiment of an ultrasound medical treatment system of the invention together with a cross section of a portion of a patient illustrated in the form of patient tissue to be thermally ablated by the system;

FIG. 2 is a block diagram of a first method of the invention for medically treating patient tissue with ultrasound which optionally can employ the embodiment of the ultrasound medical treatment system of FIG. 1;

FIG. 3 is a block diagram of a second method of the invention for medically treating patient tissue with ultrasound which optionally can employ the embodiment of the ultrasound medical treatment system of FIG. 1;

FIG. 4 is a block diagram of a third method of the invention for medically treating patient tissue with ultrasound which optionally can employ the embodiment of the ultrasound medical treatment system of FIG. 1;

FIG. 5 is a block diagram of a fourth method of the invention for medically treating patient tissue with ultrasound which optionally can employ the embodiment of the ultrasound medical treatment system of FIG. 1;

FIG. 6 is a view along lines 6-6 of FIG. 1 showing a group arrangement of elements of the array of ultrasound transducer elements of the ultrasound medical treatment transducer of FIG. 1;

FIG. 7 is a view, as in FIG. 6, but showing an alternate group arrangement of elements; and

FIG. 8 is a view, as in FIG. 6, but showing the sequential-in-position numbering of elements which, in one enablement, are sequentially-in-time activated by the controller of FIG. 1 in overlapping groups of elements.

### DETAILED DESCRIPTION OF THE INVENTION

Before explaining the present invention in detail, it should be noted that the invention is not limited in its application or use to the details of construction and arrangement of parts and/or steps illustrated in the accompanying drawings and description. The illustrative embodiment, examples, and methods of the invention may be implemented or incorporated in other embodiments, examples, methods, variations and modifications, and may be practiced or carried out in various ways. Furthermore, unless otherwise indicated, the terms and expressions employed herein have been chosen for the purpose of describing the illustrative embodiment and methods of the present invention for the convenience of the reader and are not for the purpose of limiting the invention.

It is understood that any one or more of the following-described methods, expressions of an embodiment, examples, implementations, applications, variations, modifications, etc. can be combined with any one or more of the other following-described methods, expressions of an embodiment, examples, implementations, applications, variations, modifications, etc. For example, and without limitation, the methods of the invention can be performed using the embodiment of the invention.

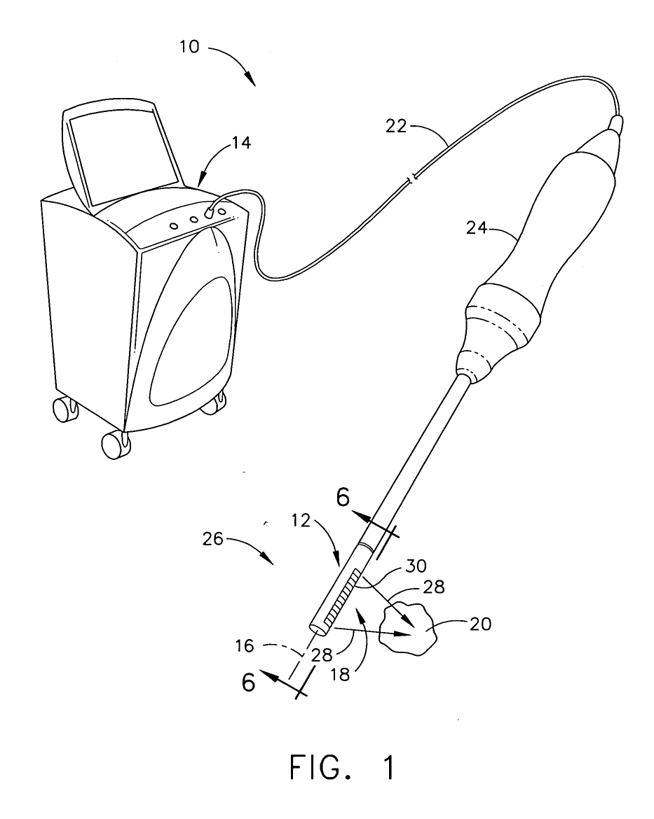

Referring now to the drawings, an embodiment of an ultrasound medical treatment system 10 is shown in FIG. 1. In a first expression of the embodiment of FIG. 1, an ultrasound medical treatment system 10 includes an ultrasound medical treatment transducer assembly 12 and a controller 14. The ultrasound medical treatment transducer assembly 12 has a longitudinal axis 16 and has an ultrasound medical treatment transducer 18. The controller 14 rotationally controls the medical treatment transducer 18 to emit ultrasound to thermally ablate (i.e., form a lesion in) patient tissue 20 for a plurality of predetermined time intervals each associated with the medical treatment transducer 18 rotationally disposed at a different one of an equal number of predetermined angular positions about the longitudinal axis 16, wherein a next-in-time time interval is associated with an angular position which is spatially non-adjacent to an angular position associated with a present-in-time time interval.

In one enablement of the first expression of the embodiment of FIG. 1, each next-in-time time interval is associated with an angular position which is spatially non-adjacent to an angular position associated with a present-in-time time interval. In one implementation of the first expression of the embodiment of FIG. 1, each time interval is substantially identical, and the angular distance between spatially adjacent angular positions is substantially identical. Other enablements and implementations are left to the artisan.

In one example of the first expression of the embodiment of FIG. 1, there are 18 angular positions, wherein the angular distance between spatially adjacent angular positions is substantially 20 degrees. The first-in-time time interval is associated with a reference angular position of 0 degrees, and sequentially-following-in-time time intervals are associated respectively with angular positions of 180, 80, 260, 140, 320, 40, 220, 100, 280, 160, 60, 240, 20, 300, 200, 120 and 340 degrees.

In one construction of the first expression of the embodiment of FIG. 1, a cable 22 operatively connects the controller 14 to the transducer 18. In one variation, the cable 18 connects the controller 14 to a handpiece 24 which is operatively connected to an end effector 26 which supports the transducer 18. In FIG. 1, the envelope of ultrasound (which is shown as a focused beam but can be an unfocused or divergent beam) from the transducer 18 is indicated by arrowed lines 28. The ultrasound medical treatment transducer 18 includes an array of ultrasound transducer elements 30. In one variation, not shown, the transducer 18 has only one transducer element.

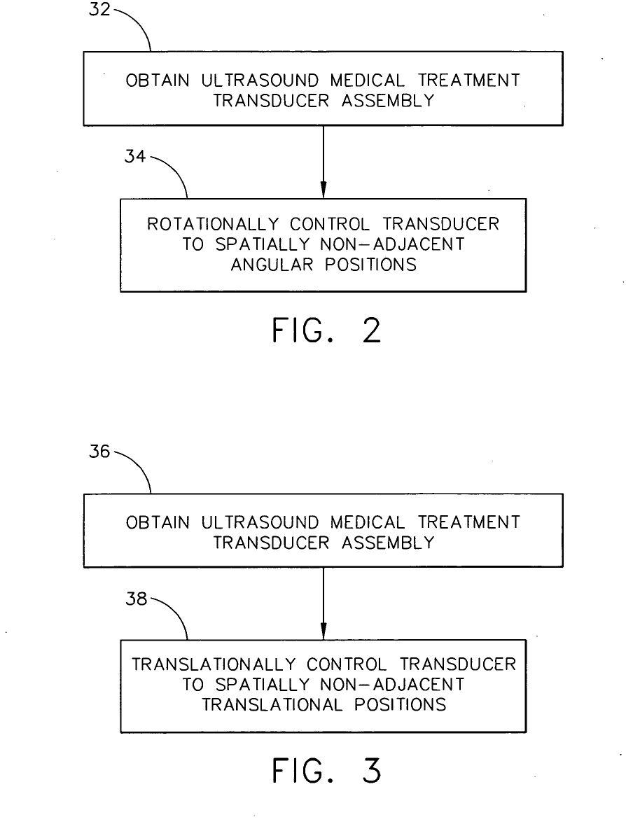

A first method of the invention is shown in block diagram form in FIG. 2 and is for medically treating patient tissue 20 with ultrasound. The first method includes steps a) through b). Step a) is labeled “Obtain Ultrasound Medical Treatment Transducer Assembly” in block 32 of FIG. 2. Step a) includes obtaining an ultrasound medical treatment transducer assembly 12 having a longitudinal axis 16 and having an ultrasound medical treatment transducer 18. Step b) is labeled “Rotationally Control Transducer To Spatially Non-Adjacent Angular Positions” in block 34 of FIG. 2. Step b) includes controlling the medical treatment transducer 18 to emit ultrasound to thermally ablate the patient tissue 20 for a plurality of predetermined time intervals each associated with the medical treatment transducer rotationally disposed at a different one of an equal number of predetermined angular positions about the longitudinal axis 16, wherein a next-in-time time interval is associated with an angular position which is spatially non-adjacent to an angular position associated with a present-in-time time interval.

In one employment of the first method of FIG. 2, a user alone in step b) effects a change in angular position of the medical treatment transducer 18, such as by the user manually rotating the medical treatment transducer 18 by rotating the handpiece 24. In another employment, a controller 14 in step b) rotationally controls the medical treatment transducer 18 to change angular position and emit ultrasound. In an additional employment, not shown, a user in step b) changes the angular position of the medical treatment transducer 18 by rotating a knob or pushing a button to activate a motor, as is within the construction skill of the artisan.

In one enablement of the first method of FIG. 2, each next-in-time time interval is associated with an angular position which is spatially non-adjacent to an angular position associated with a present-in-time time interval. In one implementation of the first method of FIG. 2, each time interval is substantially identical, and the angular distance between spatially adjacent angular positions is substantially identical. Other enablements and implementations are left to the artisan.

In one example of the first method of FIG. 2, there are 18 angular positions, wherein the angular distance between spatially adjacent angular positions is substantially 20 degrees. The first-in-time time interval is associated with a reference angular position of 0 degrees, and sequentially-following-in-time time intervals are associated respectively with angular positions of 180, 80, 260, 140, 320, 40, 220, 100, 280, 160, 60, 240, 20, 300, 200, 120 and 340 degrees.

Applicants performed a procedure on ex vivo liver tissue using a conventional treatment procedure. The ultrasound transducer had a linear-array of transducer elements and was inserted interstitially into the tissue. The transducer emitted intense ultrasound for 45 seconds in chronological order at each spatially-adjacent angular position spaced 5 degrees apart for a total transducer angular coverage of 100 degrees. The ablation depth was about 2.5 centimeters at the first angular position. However, the other angular positions had an ablation depth of only about 1 centimeter because of the ultrasound attenuation (shadowing or screening) effects caused by each previous in time and spatially-adjacent angular position.

Applicants, using an example of the first method of the invention, performed another procedure with sequentially-following-in-time time intervals associated respectively with angular positions of 180, 80, 260, 140, 320, 40, 220, 100, 280, 160, 60, 240, 20, 300, 200, 120 and 340 degrees. Applicants found a uniform lesion of about 4 centimeters in diameter was created. The results were a substantial increase in treatment depth and lesion uniformity over the conventional treatment procedure. This technique for tissue effect maximization was also validated by Applicants during in vivo tests using various transducer types and various source conditions including various time intervals and various angular positions. Applicants believe that employing non-adjacent angular positions for subsequent treatment time intervals allows more time for tissue to cool and for gas to dissipate from the current treatment angular position which substantially avoids or reduces the ultrasound-attenuation effects of the current treatment before returning to angular positions adjacent the current angular position.

In one extension of the first method of FIG. 2 (and in an extension of any or all of the following methods), step b) can be repeated as necessary, in a forward or backward spatial manner, wherein, in one implementation, the beginning of a repeated step b) is not spatially adjacent the ending of a previous step b), as can be appreciated by the artisan.

In a second expression of the embodiment of FIG. 1, an ultrasound medical treatment system 10 includes an ultrasound medical treatment transducer assembly 12 and a controller 14. The ultrasound medical treatment transducer assembly 12 has a longitudinal axis 16 and has an ultrasound medical treatment transducer 18. The controller 14 translationally controls the medical treatment transducer 18 to emit ultrasound to thermally ablate patient tissue 20 for a plurality of predetermined time intervals each associated with the medical treatment transducer 18 translationally disposed at a different one of an equal number of predetermined translational positions along the longitudinal axis 16, wherein a next-in-time time interval is associated with a translational position which is spatially non-adjacent to a translational position associated with a present-in-time time interval.

In one enablement of the second expression of the embodiment of FIG. 1, each next-in-time time interval is associated with a translational position which is spatially non-adjacent to a translational position associated with a present-in-time time interval. In one implementation of the second expression of the embodiment of FIG. 1, each time interval is substantially identical, and the translational distance between spatially adjacent translational positions is substantially identical. Other enablements and implementations are left to the artisan.

In one example of the second expression of the embodiment of FIG. 1, there are 5 translational positions, wherein the translational distance between spatially adjacent translational positions is substantially 2 millimeters. The first-in-time time interval is associated with a reference angular position of 1 millimeter from a reference translational position, and sequentially-following-in-time time intervals are associated respectively with translational positions of 7, 3, 9 and 5 millimeters from the reference translational position.

A second method of the invention is shown in block diagram form in FIG. 3 and is for medically treating patient tissue 20 with ultrasound. The second method includes steps a) through b). Step a) is labeled “Obtain Ultrasound Medical Treatment Transducer Assembly” in block 36 of FIG. 3. Step a) includes obtaining an ultrasound medical treatment transducer assembly 12 having a longitudinal axis 16 and having an ultrasound medical treatment transducer 18. Step b) is labeled “Translationally Control Transducer To Spatially Non-Adjacent Translational Positions” in block 38 of FIG. 3. Step b) includes controlling the medical treatment transducer 18 to emit ultrasound to thermally ablate the patient tissue 20 for a plurality of predetermined time intervals each associated with the medical treatment transducer translationally disposed at a different one of an equal number of predetermined translational positions along the longitudinal axis 16, wherein a next-in-time time interval is associated with a translational position which is spatially non-adjacent to a translational position associated with a present-in-time time interval.

In one employment of the second method of FIG. 3, a user alone in step b) effects a change in translational position of the medical treatment transducer 18, such as by the user manually translating the medical treatment transducer 18 by translating the handpiece 24. In another employment, a controller 14 in step b) translationally controls the medical treatment transducer 18 to change translational position and emit ultrasound. In an additional employment, not shown, a user in step b) changes the translational position of the medical treatment transducer 18 by rotating or translating a knob or pushing a button to activate a motor, as is within the construction skill of the artisan.

In one enablement of the second method of FIG. 3, each next-in-time time interval is associated with a translational position which is spatially non-adjacent to a translational position associated with a present-in-time time interval. In one implementation of the second method of FIG. 3, each time interval is substantially identical, and the translational distance between spatially adjacent translational positions is substantially identical. Other enablements and implementations are left to the artisan.

In one example of the second method of FIG. 3, there are 5 translational positions, wherein the translational distance between spatially adjacent translational positions is substantially 2 millimeters. The first-in-time time interval is associated with a reference angular position of 1 millimeter from a reference translational position, and sequentially-following-in-time time intervals are associated respectively with translational positions of 7, 3, 9 and 5 millimeters from the reference translational position.

In a third expression of the embodiment of FIG. 1, an ultrasound medical treatment system 10 includes an ultrasound medical treatment transducer assembly 12 and a controller 14. The controller 14 positionally controls the medical treatment transducer 18 to emit ultrasound to thermally ablate patient tissue 20 for a plurality of predetermined time intervals each associated with the medical treatment transducer 18 positionally disposed at a different one of an equal number of predetermined positions, wherein a next-in-time time interval is associated with a position which is spatially non-adjacent to a position associated with a present-in-time time interval.

In one example of the third expression of the embodiment of FIG. 1, the controller 14 rotationally and translationally moves the medical treatment transducer 18. In another example, the controller 14 only rotationally moves the transducer 18. In a further example, the controller 14 only translationally moves the transducer 18.

In a fourth expression of the embodiment of FIG. 1, an ultrasound medical treatment system 10 includes an ultrasound medical treatment transducer assembly 12 and a controller 14. The ultrasound medical treatment transducer assembly 12 has a longitudinal axis 16 and has an ultrasound medical treatment transducer 18. The controller 14 rotationally controls the medical treatment transducer 18 to emit ultrasound to thermally ablate patient tissue 20 for a predetermined time interval during which the medical treatment transducer 18 is substantially-continuously rotated through an angular distance about the longitudinal axis 16.

In one enablement of the fourth expression of the embodiment of FIG. 1, the medical treatment transducer is continuously rotated at a substantially constant angular speed. In one example, the angular distance is greater than 360 degrees. In one variation, the angular distance is a multiple of 360 degrees. In another example, the angular distance is less than 360 degrees.

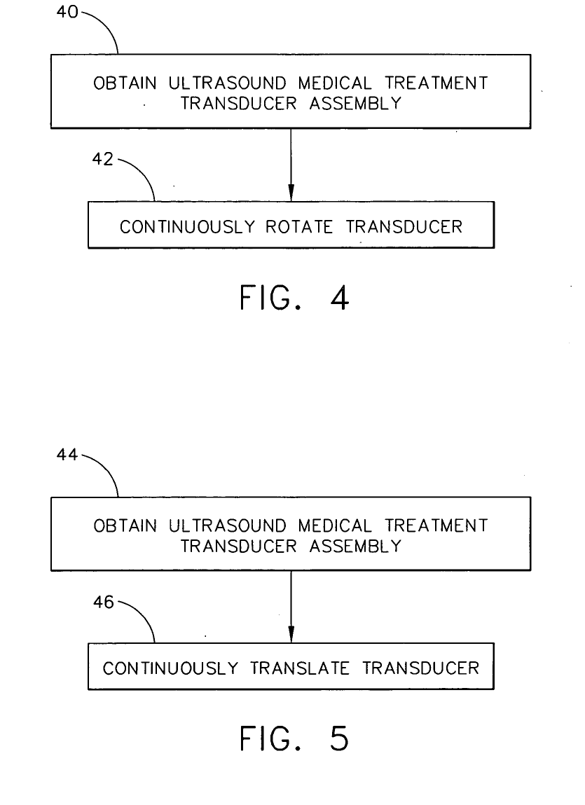

A third method of the invention is shown in block diagram form in FIG. 4 and is for medically treating patient tissue 20 with ultrasound. The third method includes steps a) through b). Step a) is labeled “Obtain Ultrasound Medical Treatment Transducer Assembly” in block 40 of FIG. 4. Step a) includes obtaining an ultrasound medical treatment transducer assembly 12 having a longitudinal axis 16 and having an ultrasound medical treatment transducer 18. Step b) is labeled “Continuously Rotate Transducer” in block 42 of FIG. 4. Step b) includes controlling the medical treatment transducer 18 to emit ultrasound to thermally ablate the patient tissue 20 for a predetermined time interval during which the medical treatment transducer is substantially-continuously rotated through an angular distance about the longitudinal axis 16.

In one enablement of the third method of FIG. 4, the medical treatment transducer is continuously rotated at a substantially constant angular speed. In one example, the angular distance is greater than 360 degrees. In one variation, the angular distance is a multiple of 360 degrees. In another example, the angular distance is less than 360 degrees.

In a fifth expression of the embodiment of FIG. 1, an ultrasound medical treatment system 10 includes an ultrasound medical treatment transducer assembly 12 and a controller 14. The ultrasound medical treatment transducer assembly 12 has a longitudinal axis 16 and has an ultrasound medical treatment transducer 18. The controller 14 translationally controls the medical treatment transducer 18 to emit ultrasound to thermally ablate patient tissue 20 for a predetermined time interval during which the medical treatment transducer 18 is substantially-continuously translated a translational distance along the longitudinal axis 16. In one example, the medical treatment transducer 18 is continuously translated at a substantially constant translational speed.

A fourth method of the invention is shown in block diagram form in FIG. 5 and is for medically treating patient tissue 20 with ultrasound. The fourth method includes steps a) through b). Step a) is labeled “Obtain Ultrasound Medical Treatment Transducer Assembly” in block 44 of FIG. 5. Step a) includes obtaining an ultrasound medical treatment transducer assembly 12 having a longitudinal axis 16 and having an ultrasound medical treatment transducer 18. Step b) is labeled “Continuously Translate Transducer” in block 46 of FIG. 5. Step b) includes controlling the medical treatment transducer 18 to emit ultrasound to thermally ablate the patient tissue 20 for a predetermined time interval during which the medical treatment transducer is substantially-continuously translated a translational distance along the longitudinal axis 16. In one example, the medical treatment transducer 18 is continuously translated at a substantially constant translational speed.

Applicants performed a procedure on ex vivo liver tissue using a conventional treatment procedure. The ultrasound transducer had a linear-array of transducer elements and was placed in front of the tissue with a standoff distance of a few millimeters. The transducer emitted intense ultrasound for 4 minutes in chronological order at each spatially-adjacent translational position spaced 18 millimeters apart. The ablation depth was about 35 millimeters at the first translational position. However, the other translational positions had an ablation depth of only about 17 millimeters because of the ultrasound attenuation (shadowing or screening) effects caused by each previous in time and spatially-adjacent translational position.

Applicants, using an example of the fourth method of the invention, performed another procedure with a transducer continuous linear translational speed of 2 millimeters per second from one side of a 53 millimeter transducer scan linearly to the other side, with returning the transducer to the starting position while therapy was off, and with repeating this sequence for 18 minutes. Applicants found a uniform lesion was created having a depth of about 31 to 34 millimeters. The results were a substantial increase in treatment depth and lesion uniformity over the conventional treatment procedure. This technique for tissue effect maximization was also validated by Applicants during in vivo tests using various transducer types and various source conditions including various translational speeds. Applicants believe that employing a transducer continuous translational speed allows more time for tissue to cool and for gas to dissipate from the current treatment position which substantially avoids or reduces the ultrasound-attenuation effects of the current treatment before returning to the same treatment position during a repeat continuously-moving scan of the transducer.

In a sixth expression of the embodiment of FIG. 1, an ultrasound medical treatment system 10 includes an ultrasound medical treatment transducer assembly 12 and a controller 14. The controller 14 positionally controls the medical treatment transducer 18 to emit ultrasound to thermally ablate patient tissue 20 for a predetermined time interval during which the medical treatment transducer 18 substantially-continuously changes position.

In one example of the sixth expression of the embodiment of FIG. 1, the controller 14 rotationally and translationally moves the medical treatment transducer 18. In another example, the controller 14 only rotationally moves the transducer 18. In a further example, the controller 14 only translationally moves the transducer 18.

In a seventh expression of the embodiment of FIG. 1, an ultrasound medical treatment system 10 includes an ultrasound medical treatment transducer 18 and a controller 14. The medical treatment transducer 18 has an array of ultrasound transducer elements 30 and has a multiplicity of element groups each including at least one ultrasound transducer element 30 of the array, wherein each ultrasound transducer element 30 of the array belongs to only one element group (i.e., the groups do not overlap). The controller 14 controls the medical treatment transducer 18 to emit ultrasound to thermally ablate patient tissue 20 for a plurality of predetermined time intervals each associated with emitting ultrasound from a different one of the element groups. In one arrangement, each element group has an equal number of ultrasound transducer elements 30.

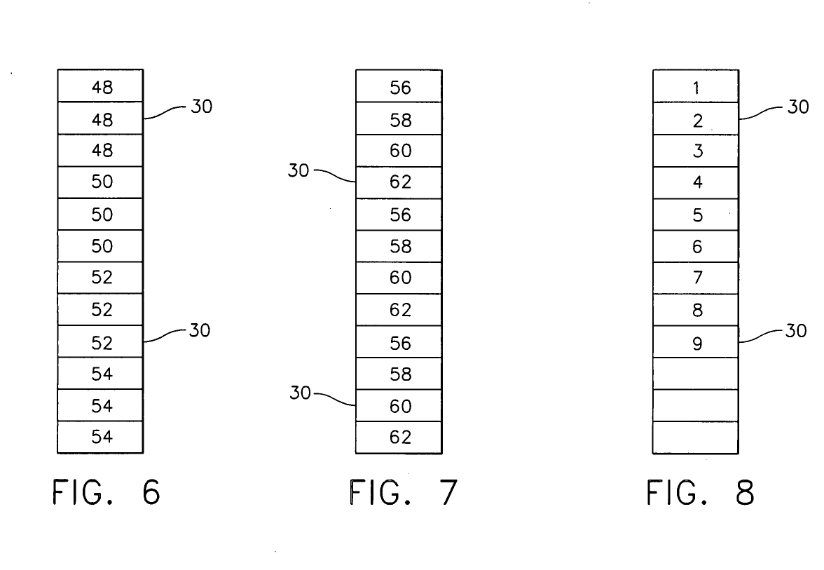

In a first construction of the seventh expression of the embodiment of FIG. 1, as seen in FIG. 6, the array is a linear array of ultrasound transducer elements 30 (wherein each element 30 is depicted as a box with several boxes having lead lines leading to a number 30). All of the ultrasound transducer elements 30 of an element group 48, 50, 52 and 54 (wherein group 40 consists of those transducer elements 30 having a number 48 within a box, group 50 consists of those transducer elements 30 having a number 50 within a box, etc.) are adjacent at least one other ultrasound transducer element 30 of that element group 48, 50, 52 and 54. All but two of the ultrasound transducer elements 30, for element groups 48, 50, 52 and 54 having at least three ultrasound transducer elements 30, are adjacent two other ultrasound transducer elements 30 of that element group 48, 50, 52 and 54.

In a first variation of the first construction of the seventh expression of the embodiment of FIG. 1, each next-in-time time interval is associated with an element group 48, 50, 52 and 54 which is spatially non-adjacent the element group 48, 50, 52 and 54 associated with a present-in-time time interval. In a different variation, each next-in-time time interval is associated with an element group 48, 50, 52 and 54 which is spatially adjacent the element group 48, 50, 52 and 54 associated with a present-in-time time interval.

In a second construction of the seventh expression of the embodiment of FIG. 1, as seen in FIG. 7, the array is a linear array of ultrasound transducer elements 30 (wherein each element 30 is depicted as a box with several boxes having lead lines leading to a number 30). No ultrasound transducer element 30 of an element group 56, 58, 60 and 62 (wherein group 56 consists of those transducer elements 30 having a number 56 within a box, group 58 consists of those transducer elements 30 having a number 58 within a box, etc.) is adjacent any other ultrasound transducer element 30 of that element group 48, 50, 52 and 54.

In one variation of the seventh expression of the embodiment of FIG. 1, the ultrasound transducer elements 30 are electronically controlled by the controller 14 to change the focus and/or the beam angle of the ultrasound emitted by the ultrasound medical treatment transducer 18.

In an eighth expression of the embodiment of FIG. 1, an ultrasound medical treatment system 10 includes an ultrasound medical treatment transducer 18 and a controller 14. The medical treatment transducer 18 has an array of ultrasound transducer elements 30, wherein the ultrasound transducer elements 30 are disposed substantially along a straight or curved line. The controller 14 controls the medical treatment transducer 18 to emit ultrasound to thermally ablate patient tissue by sequentially-in-time activating positionally-overlapping groups of sequential-in-position ultrasound transducer elements 30.

In one employment of the eighth expression of the embodiment of FIG. 1, as seen in FIG. 8, the array includes sequential-in-position ultrasound transducer elements 30 numbered 1, 2, 3, . . . N. In one example of this employment, the controller 14 first only activates ultrasound transducer elements 30 numbered 1 through 8, then only activates ultrasound transducer elements 30 numbered 2 through 9, . . . , and then only activates ultrasound transducer elements 30 numbered N minus 7 through N. It is noted that N is 12 in FIG. 8, but N can be any number. In FIG. 8, the top ultrasound transducer element 30 is numbered 1 in the box depicting that element, the next from the top is numbered 2, etc. wherein only nine have been numbered for clarity. In another employment and example, not shown, the controller 14 first only activates ultrasound transducer elements 30 numbered 1 through 10, then only activates ultrasound transducer elements 30 numbered 6 through 15, then only activates ultrasound transducer elements 30 numbered 11 through 20, etc. Other employments and examples are left to the artisan.

In one construction of the eighth expression of the embodiment of FIG. 1, not shown, the ultrasound medical treatment transducer has one or more additional similar or identical arrays of ultrasound transducer elements aligned with the previously-described array. Other constructions are left to those skilled in the art. In one variation of the eighth expression of the embodiment of FIG. 1, the ultrasound transducer elements 30 are electronically controlled by the controller 14 to change the focus and/or the beam angle of the ultrasound emitted by the ultrasound medical treatment transducer 18.

Several benefits and advantages are obtained from one or more of the expressions of the embodiment and/or the methods of the invention. Applicants found having temporally-adjacent ablation time intervals be associated with spatially non-adjacent transducer positions substantially avoids or reduces transient, ultrasound-caused, ultrasound-attenuating effects (from tissue cavitation, tissue boiling, and/or temperature-related increases in tissue ultrasonic absorption) found near conventionally stepwise just-treated spatially adjacent tissue. This increased treatment depth and achieved a more uniform thermal lesion.

Applicants also found substantially-continuously moving the ultrasound medical treatment transducer substantially avoids or reduces transient, ultrasound-caused, ultrasound-attenuating effects (from tissue cavitation, tissue boiling and/or temperature-related increases in tissue ultrasonic absorption) found near conventionally stepwise just-treated spatially adjacent tissue. This increased treatment depth and achieved a more uniform thermal lesion.

Applicants believe that using different transducer element groups (of a medical treatment transducer having an array of transducer elements) for predetermined time intervals, wherein each element belongs to only one element group, or sequentially-in-time activating positionally-overlapping groups of sequential-in-position ultrasound transducer elements, should also substantially avoid or reduce transient, ultrasound-caused, ultrasound-attenuating effects (e.g., from tissue cavitation, tissue boiling and/or temperature-related increases in tissue ultrasonic absorption) found near conventionally stepwise just-treated spatially adjacent tissue. This should increase treatment depth and achieve a more uniform thermal lesion.

Thus, one or more of the methods or expressions of the embodiment of the invention should result in more consistent lesion size and quality across different tissue properties, geometries, and ultrasonic source conditions, and the resulting reduction of ultrasound-attenuating effects (e.g., screening and shadowing ultrasound effects) should allow greater treatment depths, shorter treatment times, and/or the formation of more regular and controllable (and therefore more spatially selective) thermal lesions.

While the present invention has been illustrated by a description of several methods and several expressions of an embodiment, it is not the intention of the applicants to restrict or limit the spirit and scope of the appended claims to such detail. Numerous other variations, changes, and substitutions will occur to those skilled in the art without departing from the scope of the invention. For instance, the ultrasound methods and system embodiment of the invention have application in robotic assisted surgery taking into account the obvious modifications of such method, system embodiment and components to be compatible with such a robotic system. It will be understood that the foregoing description is provided by way of example, and that other modifications may occur to those skilled in the art without departing from the scope and spirit of the appended Claims.

1. An ultrasound medical treatment system comprising:
a) an ultrasound medical treatment transducer assembly having a longitudinal axis and having an ultrasound medical treatment transducer; and
b) a controller which rotationally controls the medical treatment transducer to emit ultrasound to thermally ablate patient tissue for a plurality of predetermined time intervals each associated with the medical treatment transducer rotationally disposed at a different one of an equal number of predetermined angular positions about the longitudinal axis, wherein a next-in-time time interval is associated with an angular position which is spatially non-adjacent to an angular position associated with a present-in-time time interval.

2. The ultrasound medical treatment system of claim 1, wherein each next-in-time time interval is associated with an angular position which is spatially non-adjacent to an angular position associated with a present-in-time time interval.

3. The ultrasound medical treatment system of claim 2, wherein each time interval is substantially identical, and wherein the angular distance between spatially adjacent angular positions is substantially identical.

4. The ultrasound medical treatment system of claim 3, wherein there are 18 angular positions, wherein the angular distance between spatially adjacent angular positions is substantially 20 degrees, wherein the first-in-time time interval is associated with a reference angular position of 0 degrees, and wherein sequentially-following-in-time time intervals are associated respectively with angular positions of 180, 80, 260, 140, 320, 40, 220, 100, 280, 160, 60, 240, 20, 300, 200, 120 and 340 degrees.

5. A method for medically treating patient tissue with ultrasound comprising the steps of:
a) obtaining an ultrasound medical treatment transducer assembly having a longitudinal axis and having an ultrasound medical treatment transducer; and
b) controlling the medical treatment transducer to emit ultrasound to thermally ablate the patient tissue for a plurality of predetermined time intervals each associated with the medical treatment transducer rotationally disposed at a different one of an equal number of predetermined angular positions about the longitudinal axis, wherein a next-in-time time interval is associated with an angular position which is spatially non-adjacent to an angular position associated with a present-in-time time interval.

6. The method of claim 5, wherein each next-in-time time interval is associated with an angular position which is spatially non-adjacent to an angular position associated with a present-in-time time interval.

7. The method of claim 6, wherein each time interval is substantially identical, and wherein the angular distance between spatially adjacent angular positions is substantially identical.

8. The method of claim 7, wherein there are 18 angular positions, wherein the angular distance between spatially adjacent angular positions is substantially 20 degrees, wherein the first-in-time time interval is associated with a reference angular position of 0 degrees, and wherein sequentially-following-in-time time intervals are associated respectively with angular positions of 180, 80, 260, 140, 320, 40, 220, 100, 280, 160, 60, 240, 20, 300, 200, 120 and 340 degrees.

9. An ultrasound medical treatment system comprising:
a) an ultrasound medical treatment transducer assembly having a longitudinal axis and having an ultrasound medical treatment transducer; and
b) a controller which translationally controls the medical treatment transducer to emit ultrasound to thermally ablate patient tissue for a plurality of predetermined time intervals each associated with the medical treatment transducer translationally disposed at a different one of an equal number of predetermined translational positions along the longitudinal axis, wherein a next-in-time time interval is associated with a translational position which is spatially non-adjacent to a translational position associated with a present-in-time time interval.

10. The ultrasound medical treatment system of claim 9, wherein each next-in-time time interval is associated with a translational position which is spatially non-adjacent to a translational position associated with a present-in-time time interval.

11. The ultrasound medical treatment system of claim 10, wherein each time interval is substantially identical, and wherein the translational distance between spatially adjacent translational positions is substantially identical.

12. The ultrasound medical treatment system of claim 11, wherein there are 5 translational positions, wherein the translational distance between spatially adjacent translational positions is substantially 2 millimeters, wherein the first-in-time time interval is associated with a translational position of 1 millimeter from a reference translational position, and wherein sequentially-following-in-time time intervals are associated respectively with translational positions of 7, 3, 9 and 5 millimeters from the reference translational position.

13. A method for medically treating patient tissue with ultrasound comprising the steps of:
a) obtaining an ultrasound medical treatment transducer assembly having a longitudinal axis and having an ultrasound medical treatment transducer; and
b) controlling the medical treatment transducer to emit ultrasound to thermally ablate the patient tissue for a plurality of predetermined time intervals each associated with the medical treatment transducer translationally disposed at a different one of an equal number of predetermined translational positions along the longitudinal axis, wherein a next-in-time time interval is associated with a translational position which is spatially non-adjacent to a translational position associated with a present-in-time time interval.

14. The method of claim 13, wherein each next-in-time time interval is associated with a translational position which is spatially non-adjacent to a translational position associated with a present-in-time time interval.

15. The method of claim 14, wherein each time interval is substantially identical, and wherein the translational distance between spatially adjacent translational positions is substantially identical.

16. The method of claim 15, wherein there are 5 translational positions, wherein the translational distance between spatially adjacent translational positions is substantially 2 millimeters, wherein the first-in-time time interval is associated with a translational position of 1 millimeter from a reference translational position, and wherein sequentially-following-in-time time intervals are associated respectively with translational positions of 7, 3, 9 and 5 millimeters from the reference translational position.

17. An ultrasound medical treatment system comprising:
a) an ultrasound medical treatment transducer; and
b) a controller which positionally controls the medical treatment transducer to emit ultrasound to thermally ablate patient tissue for a plurality of predetermined time intervals each associated with the medical treatment transducer positionally disposed at a different one of an equal number of predetermined positions, wherein a next-in-time time interval is associated with a position which is spatially non-adjacent to a position associated with a present-in-time time interval.

18. An ultrasound medical treatment system comprising:
a) an ultrasound medical treatment transducer assembly having a longitudinal axis and having an ultrasound medical treatment transducer; and
b) a controller which rotationally controls the medical treatment transducer to emit ultrasound to thermally ablate patient tissue for a predetermined time interval during which the medical treatment transducer is substantially-continuously rotated through an angular distance about the longitudinal axis.

19. The ultrasound medical treatment system of claim 18, wherein the medical treatment transducer is continuously rotated at a substantially constant angular speed.

20. The ultrasound medical treatment system of claim 18, wherein the angular distance is greater than 360 degrees.

21. The ultrasound medical treatment system of claim 20, wherein the angular distance is a multiple of 360 degrees.

22. The ultrasound medical treatment system of claim 18, wherein there the angular distance is less than 360 degrees

23. A method for medically treating patient tissue with ultrasound comprising the steps of:
a) obtaining an ultrasound medical treatment transducer assembly having a longitudinal axis and having an ultrasound medical treatment transducer; and
b) controlling the medical treatment transducer to emit ultrasound to thermally ablate the patient tissue for a predetermined time interval during which the medical treatment transducer is substantially-continuously rotated through an angular distance about the longitudinal axis.

24. The method of claim 23, wherein the medical treatment transducer is continuously rotated at a substantially constant angular speed.

25. The method of claim 23, wherein the angular distance is greater than 360 degrees.

26. The method of claim 25, wherein the angular distance is a multiple of 360 degrees.

27. The method of claim 23, wherein there the angular distance is less than 360 degrees.

28. An ultrasound medical treatment system comprising:
a) an ultrasound medical treatment transducer assembly having a longitudinal axis and having an ultrasound medical treatment transducer; and
b) a controller which translationally controls the medical treatment transducer to emit ultrasound to thermally ablate patient tissue for a predetermined time interval during which the medical treatment transducer is substantially-continuously translated a translational distance along the longitudinal axis.

29. The ultrasound medical treatment system of claim 28, wherein the medical treatment transducer is continuously translated at a substantially constant translational speed.

30. A method for medically treating patient tissue with ultrasound comprising the steps of:
a) obtaining an ultrasound medical treatment transducer assembly having a longitudinal axis and having an ultrasound medical treatment transducer; and
b) controlling the medical treatment transducer to emit ultrasound to thermally ablate the patient tissue for a predetermined time interval during which the medical treatment transducer is substantially-continuously translated a translational distance along the longitudinal axis.

31. The method of claim 30, wherein the medical treatment transducer is continuously translated at a substantially constant translational speed.

32. An ultrasound medical treatment system comprising:
a) an ultrasound medical treatment transducer; and
b) a controller which positionally controls the medical treatment transducer to emit ultrasound to thermally ablate patient tissue for a predetermined time interval during which the medical treatment transducer substantially-continuously changes position.

33. An ultrasound medical treatment system comprising:
a) an ultrasound medical treatment transducer having an array of ultrasound transducer elements and having a multiplicity of element groups each including at least one ultrasound transducer element of the array, wherein each ultrasound transducer element of the array belongs to only one element group; and
b) a controller which controls the medical treatment transducer to emit ultrasound to thermally ablate patient tissue for a plurality of predetermined time intervals each associated with emitting ultrasound from a different one of the element groups.

34. The ultrasound medical treatment system of claim 33, wherein each element group has an equal number of ultrasound transducer elements.

35. The ultrasound medical treatment system of claim 33, wherein the array is a linear array of ultrasound transducer elements, wherein all of the ultrasound transducer elements of an element group are adjacent at least one other ultrasound transducer element of that element group, and wherein all but two of the ultrasound transducer elements, for element groups having at least three ultrasound transducer elements, are adjacent two other ultrasound transducer elements of that element group.

36. The ultrasound medical treatment system of claim 35, wherein each next-in-time time interval is associated with an element group which is spatially non-adjacent the element group associated with a present-in-time time interval.

37. The ultrasound medical treatment system of claim 35, wherein each next-in-time time interval is associated with an element group which is spatially adjacent the element group associated with a present-in-time time interval.

38. The ultrasound medical treatment system of claim 33, wherein the array is a linear array of ultrasound transducer elements, and wherein no ultrasound transducer element of an element group is adjacent any other ultrasound transducer element of that element group.

39. An ultrasound medical treatment system comprising:
a) an ultrasound medical treatment transducer having an array of ultrasound transducer elements, wherein the ultrasound transducer elements are disposed substantially along a straight or curved line; and
b) a controller which controls the medical treatment transducer to emit ultrasound to thermally ablate patient tissue by sequentially-in-time activating positionally-overlapping groups of sequential-in-position ultrasound transducer elements.

40. The ultrasound medical treatment system of claim 39, wherein the array includes sequential-in-position ultrasound transducer elements numbered 1, 2, 3, . . . N, wherein the controller first only activates ultrasound transducer elements numbered 1 through 8, then only activates ultrasound transducer elements numbered 2 through 9, . . . , and then only activates ultrasound transducer elements numbered N minus 7 through N.
